(19) United States
(12) Plant Patent Application Publication
(10) Pub. No.: US 2006/0059593 P1
(43) Pub. Date: Mar. 16, 2006

(54) Tree named 'xTaxodiomera peizhongii'

(76) Inventors: Ye Zengji, Shanghai (CN); Shen Lieying, Shanghai (CN); Pan Shihua, Shanghai (CN); Zhu Weijie, Shanghai (CN); Niu Huijuan, Shanghai (CN)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 10/941,465

(22) Filed: Sep. 15, 2004

(51) Int. Cl. A01H 5/00

(52) U.S. Cl. PLT/216

(57) ABSTRACT

×Taxodiomera peizhongii is a distinct and new above ground nontubular propagated cultivar comprising a tall semi-indeciduous arbor tree providing a high view. ×Taxodiomera peizhongii is well suited for afforestation in the city and has many good properties such as fast growth, wide adaptability and strong stress resistance. Its main characteristics include: (1) its base of stem is round and regular without buttress roots; (2) its bark cracks into flakes; (3) there are several main crotches five to eight meters above ground, and its canopy is nearly elliptic shape; (4) there are only male conglobate flower and no female conglobate fruit on the adult tree, and it cannot reproduce with sexual propagation manner. It possesses additional good properties including enhanced saline tolerance (salt content is below about 3.9%), alkali tolerance (7≦pH<8.9), moisture resistance and good (pleasing visual) landscape effect.

Plant named ‘Dongfangshan’

Latin name: ×Taxodiomera peizhongii

Variety denomination: Dongfangshan

Inventors: Ye Zengji, Shen Lieying, Pan Shihua, Zhu Weijie, Niu Huijuan

### BACKGROUND OF THE NEW CULTIVAR

1. Field of the Invention

The new cultivar, ×Taxodiomera peizhongii, is the filial generation of Taxodium mucronatum Ten and Crytomeria fortunei.

Cultivated plant seedlings have been asexually reproduced and have proved to be stable and uniform and have characteristics distinct from known plants. Seedling plants were found in a cultivated state. Unique characteristics of this new cultivar as herein described are firmly fixed and retain the identical distinguishing characteristics of the new cultivar through successive generations of asexual propagation. This cultivar has use in afforestation, establishing a forest network or a tree coastal shelter belt and for landscaping along rivers, in parks and in communities.

2. The Description of ×Taxodiomera Peizhongii's Particularity Relative to Its Parent

×Taxodiomera peizhongii belongs to Taxodium of Taxo diaceae, which originated from the wide cross of Taxodium mucronatum Ten (♀) and Crytomeria fortunei (♂). The main configuration of ×Taxodiomera peizhongii is similar to maternal taxodium mucronatum ten, the phenomenon of biased maternal inheritance of ×Taxodiomera peizhongii is also approved by RAPD analysis, the analysis makes it clear that the genetic distance (0.210˜0.246) between ×Taxodiomera peizhongii and maternal taxodium mucronaturn ten is far less than the genetic distance (0.757˜0.764) between it and paternal Crytomeria fortunei. Its main characters are: (1) its base of stem is round and regular without buttress root, close to paternal Crytomeria fortunei, whereas its maternal taxodium mucronatum ten has buttress root; (2) the bark of taxodim mucronatum ten cracks into strips, but the ×Taxodiomera peizhongii has transverse cracks besides split cracks and its bark cracks into flakes; (3) there often are several main crotches five to eight meters above ground in the ×Taxodiomera peizhongii's trunk stem, its canopy is nearly elliptic shape, this is obviously differ from its maternal and paternal plant, there is obviously no crotch in the trunk stem of maternal taxodim mucronatum ten, and the canopy extends to wide conic shape, however the canopy of Crytomeria fortunei is long elliptic shape in common; (4) there are only male conglobate fruit on the adult tree of more than 30 years old, arranging in vertical disposition, see the type specimen (picture 2). and no female conglobate fruit on the adult tree, and it cannot reproduce with sexual propagation manner.

Origin and Asexual Reproduction of the New Cultivar

Taxodium mucronatum Ten belongs to Taxoduim of Taxodiaceae and is a tall deciduous or semi-indeciduous arbor species, which originated in Mexico and in Texas and was introduced to the Peoples Republic of China in about 1925. The Crytomeria fortunei is belong to Cryptomeria D. Don of taxodiaceae, it is evergreen tall arbor, and is originated in Zhejiang Province of China. In 1962, after pollinating Taxodium mucronatum Ten at Nanjing Engineering College using pollen of Crytomeria fortunei, we obtained three cones. By seeding them, we were able to produce twelve seedlings. We selected five of them after planting. More than 6,000 seedlings were obtained in 1972 after stem planting.

×Taxodium peizhongii has been spread and planted experimentally after thirty years of studies conducted by Shanghai Central Station of Forestry including afforestation, cultivation and propagation tests (asexual reproduction in a cultivated state) under different ecological conditions. Such studies included detailed research on its germplasm characters and its ecological value. Presently there is a ×Taxodiomera peizhongii population of about 2,000 thirty-year-old trees in the Peoples Republic of China with the tree population growing on land having a salt content of 3.9%. Some of that tree population is growing with the base and root system submerged in water, while other trees of that population grow under various ecological areas such as riversides and parks.

Through Genetic Comparing the ×Taxodiomera Peizhongii Is Proved to Be a New Steady Breed

In the whole Chuansha forestry center with more than 1000 trunks ×Taxodiomera peizhongii, its stand forest structure is accordant.

After three continuous years observation on-site and collecting in the collecting sites of Songjiang Drunk-white Pool, Xinqiao Nursery, Jinshan Shore Park, the plant area of Petrochemical Complex, the double bank of Chuanyang River in Pudong New Area, Yangjing Nursery, Chuansha Forestry Centre and other sites of Najing and Zhenjiang, it is shown that there is no difference in the configuration between different sites.

In recent years, the materials gathered from the collecting sites of Songjiang Drunk-white Pool, Xinqiao Nursery, Jinshan Shore Park, the plant area of Petrochemical Complex, the double bank of Chuanyang River in Pudong New Area, Yangjing Nursery, Chuansha Forestry Centre and other sites of Najing and Zhenjiang are accordant, all no female conglobate fruit, there is no different instance relative to type specimen The stem structures, branch characters and canopy shapes are all comparatively accordant.

The Detailed Examples of Asexual Propagation of ×Taxodiomera peizhongii

We researched the approach of asexual propagation of ×Taxodiomera peizhongii, the specific methods are as follows: hardwood cutting, greenwood cutting, tissue culture, graft and etc., it is proved through practicing that greenwood cutting and graft are the better methods.

Greenwood Cutting

Use the current growth half-lignified shoot of ×Taxodiomera peizhongii to be the cutting wood, cutting in the seasons of early summer (plum season), midsummer and early autumn, furthermore, can cut in the winter utilizing the establishment of greenhouse. Among them, the effects of cutting in plum season and winter are the best, the rooting rate can reach 72%˜75%, higher than the rooting rate of cutting in midsummer and early autumn.

Specific Method:

Cut the shoot into a cutting wood of 10˜12 cm with a sharp knife, the notch must be smooth. Before cut, the cutting wood should be dipped rapidly into naphthyl acetic acid of 500 ppm or dipped in naphthylacetic acid of 50 ppm for 6 hours, the cutting wood must be cut in medium (coarse yellow sand, plant ash, pearl sand or roseite) by ⅔ or ½. After cutting, compress tightly the medium, water completely and do the treatment of shading and wetting, it can root after 40˜60 days well managing like this.

Grafting

Graft in early spring before foliation.

Use the seedling of annual taxodium ascendens Brongn or taxodium distichum as tree stock, take the vigorous annual shoot without diseases and insect pests as scion wood, and trim the scion wood to be a scion with only 2˜3 buds, adopt the method of “heart center approach grafting”, when the graft is surviving, do the stock tip pruning, but remain a preponderant bud on the scion wood, and well foster it, therewith its growth can be more than 1M.

### SUMMARY OF THE INVENTION

Distinctive characteristics of ×Taxodiomera peizhongii include: (1) its base of stem is round and regular without buttress root; (2) its bark cracks into flakes (3) there are several main crotches five to eight meters above ground, (4) its canopy is nearly elliptic shape; (5) there are only male conglobate flower and no female conglobate fruit on the adult tree, and (6) it only reproduces asexually. ×Taxodiomera peizhongii also possesses additional good properties including saline tolerance (salt content is below 3.9%), alkali tolerance (7≦pH≦8.9), moisture resistance and provides good landscape effect. ×Taxodiomera peizhongii is suitable for afforestation and establishing farmland, forest network, and coastal shelter belt on intertidal zones. ×Taxodiomera peizhongii grows well in alkali soils, alkaline lands, marsh, riverbanks, gardens, park landscapes, factories and communities.

×Taxodiomera peizhongii has been successfully exactly reproduced from its originating as a seedling using asexual reproduction methods such as twig cuttage, a asexual reproduction produces an exact copy of the ×Taxodiomera peizhongii plant which is identical to the original plant in every distinguishing characteristic.

### BRIEF DESCRIPTION OF THE PHOTOGRAPHS

FIG. 1 shows the biggest ×Taxodiomera peizhongii tree, a thirty-year-old tree growing in Chuansha Forestry Center, Shanghai, Peoples Republic of China. Its diameter at breast height is 62.5 cm. Its stem requires two persons to fully clasp hands to reach around the perimeter of the tree.

FIG. 2 shows a specimen of ×Taxodiomera peizhongii.

FIG. 3 shows one of the characteristics of ×Taxodiomera peizhongii—round and regular stem base. The stem base of ×Taxodiomera peizhongii is round and regular without buttress roots.

FIG. 4 shows Taxodium mucronatum Ten, the control variety of ×Taxodiomera peizhongii, which has distinct buttress roots.

FIG. 5 shows a second characteristic of ×Taxodiomera peizhongii—its flaky bark.

FIG. 6 shows a third characteristic of ×Taxodiomera peizhongii—multiple crotches on the trunk. There are always several crotches on the trunk five to eight meters above ground.

FIG. 7 shows a semi-indeciduous characteristic of ×Taxodiomera peizhongii. ×Taxodiomera peizhongii is lush and it foliage begins to fall off at the end of the calendar month December, while the foliage of metasequoia falls off after November.

FIG. 8 shows the moisture tolerance characteristic of ×Taxodiomera peizhongii. In Songjiang Xinqiao Xinjie Nursery of Shanghai city, several scores of ×Taxodiomera peizhongii have grown for almost twenty years in the river due to the collapse of some of the riverbank which occurred in the 1980's. The growth vigor of these trees growing in the river is comparable to those trees which remained growing on a remaining part of the riverbank.

FIG. 9 shows saline and alkali tolerance characteristics of ×Taxodiomera peizhongii. Jinshan Binhai Park in Shanghai city is reclaimed beach land. Even though salt concentration of this sloping forest land reaches 3.9%, ×Taxodiomera peizhongii still grows.

FIG. 10 shows a landscape view of the biggest ×Taxodiomera peizhongii forest in China—×Taxodiomera peizhongii forest in Chuansha Forestry Center. Chuansha Forestry Center is located on one of the main pathways of typhoon(s) from the sea to Shanghai city. The pH value of the forestry center soil is about 8.6. However, ×Taxodiomera peizhongii grows very well in this alkali soil.

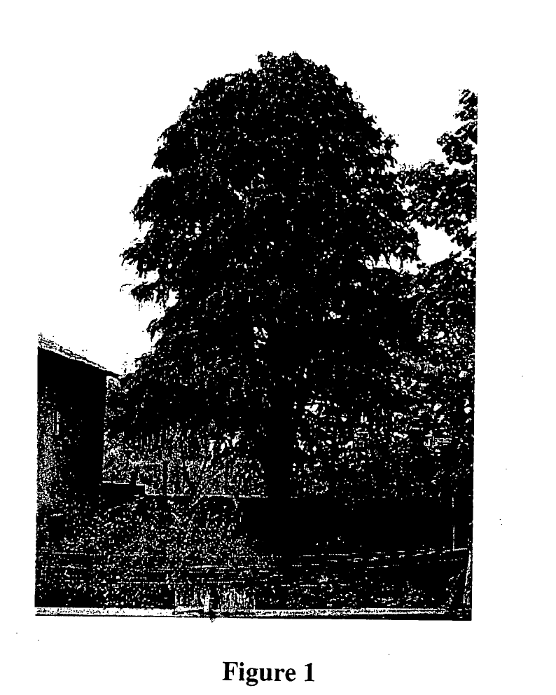

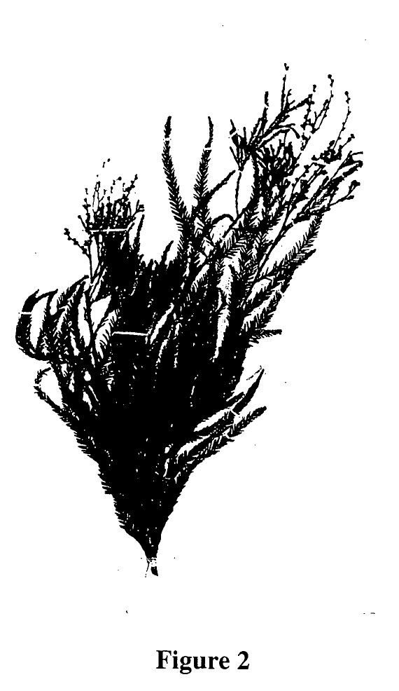

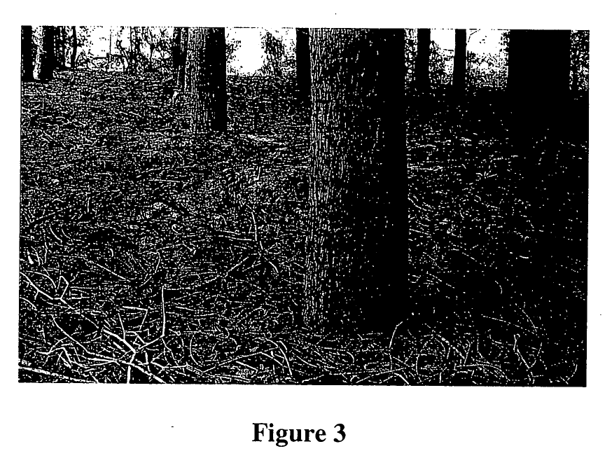

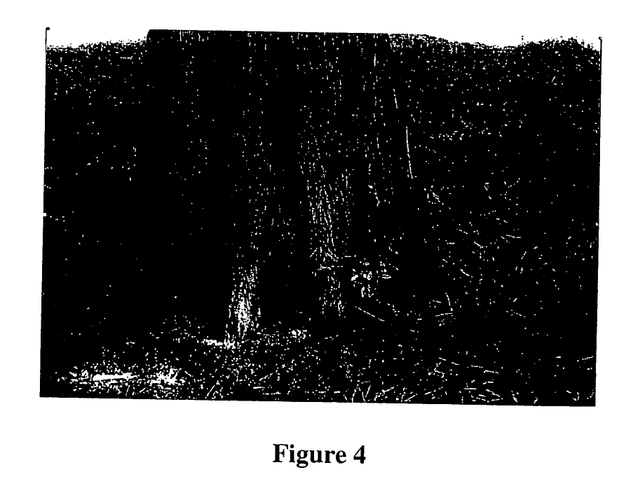

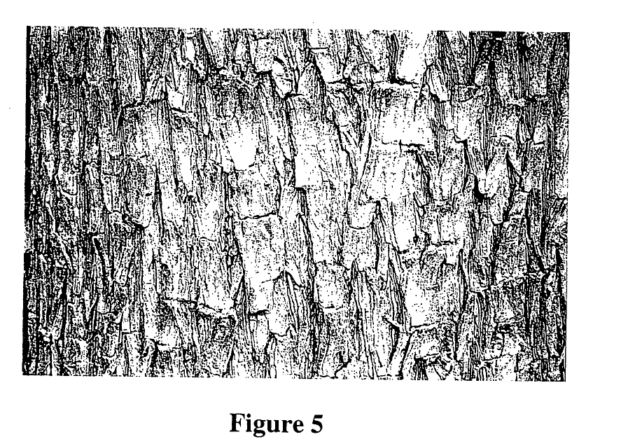

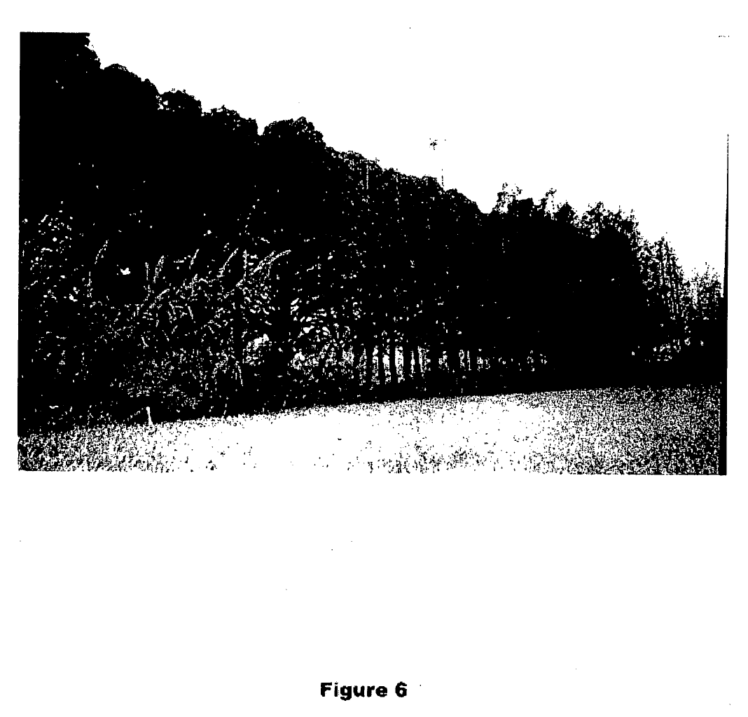

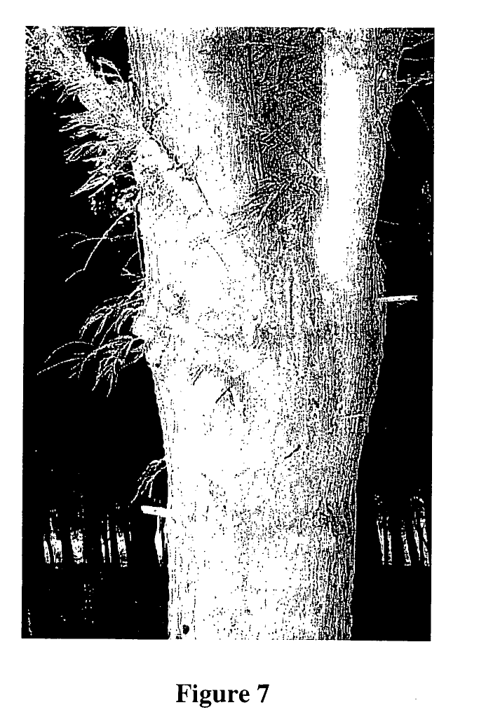

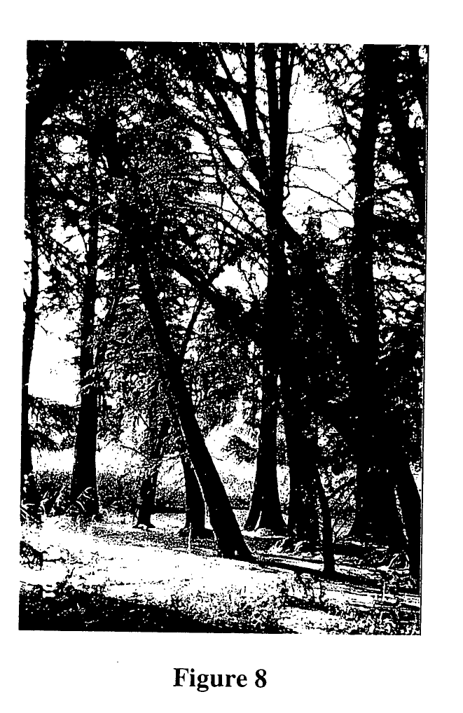

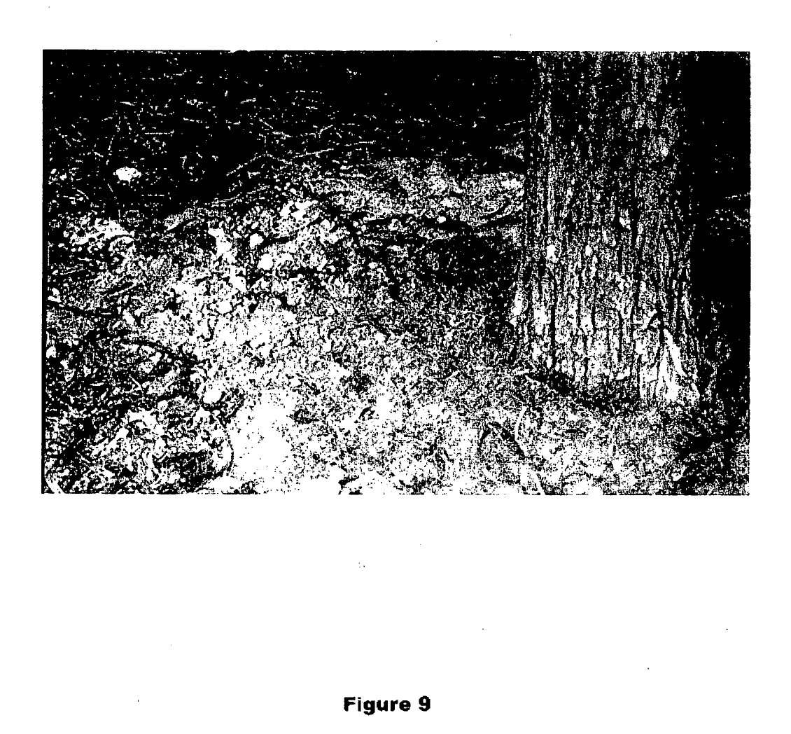

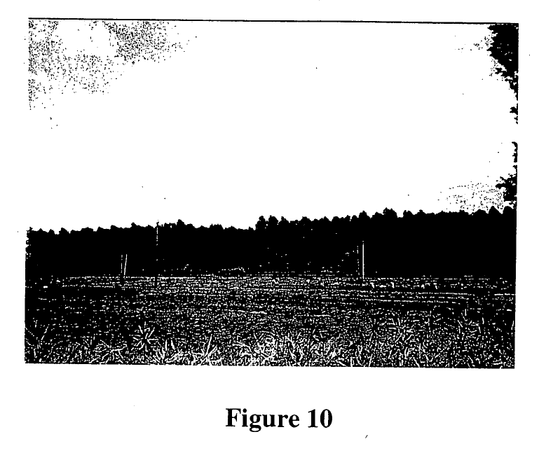

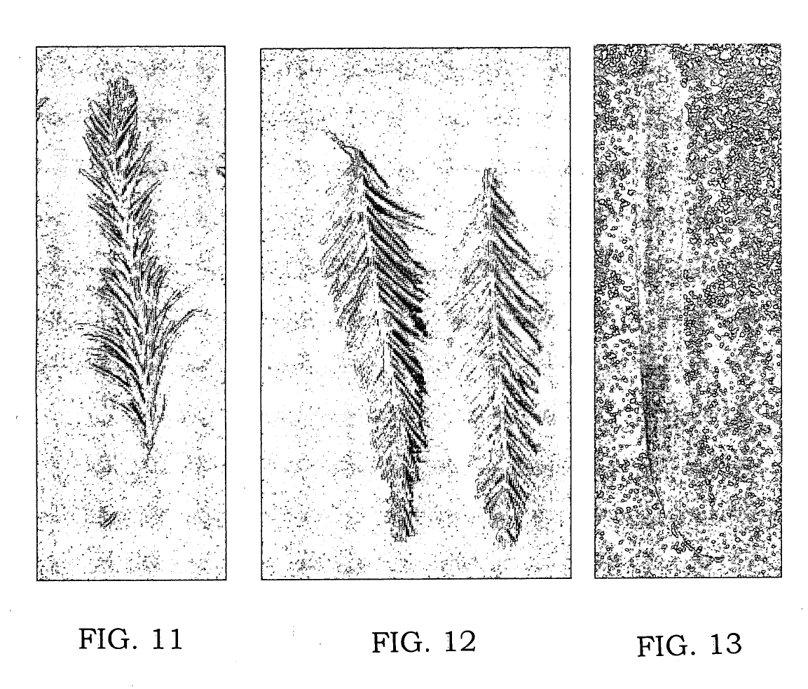

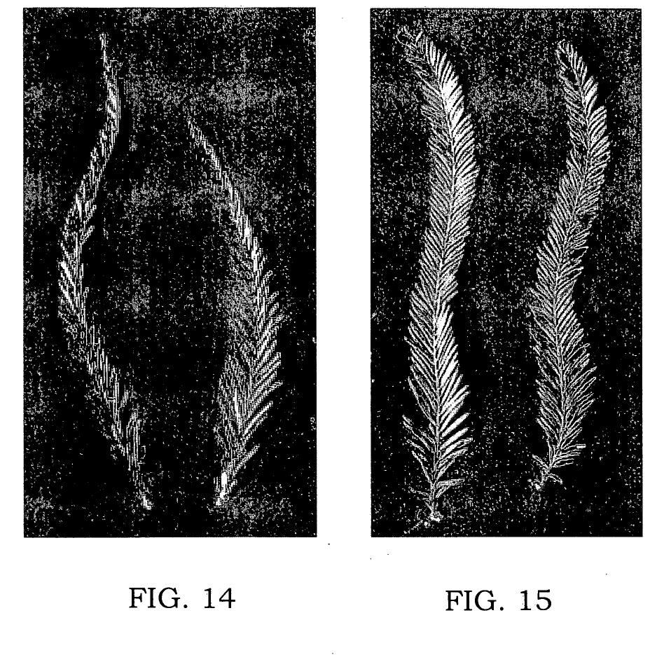

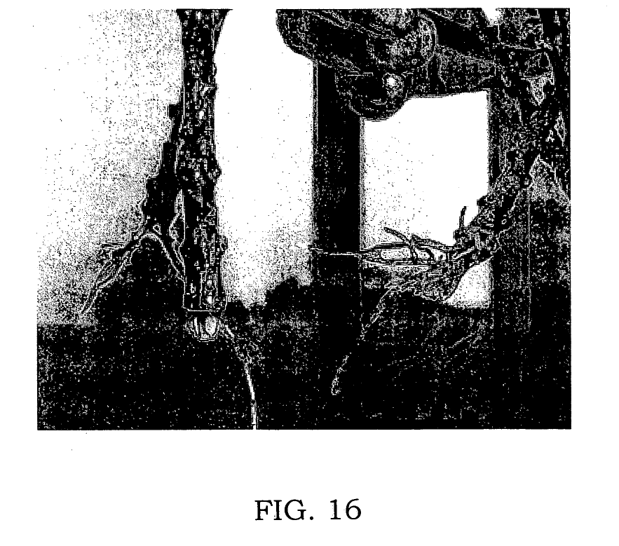

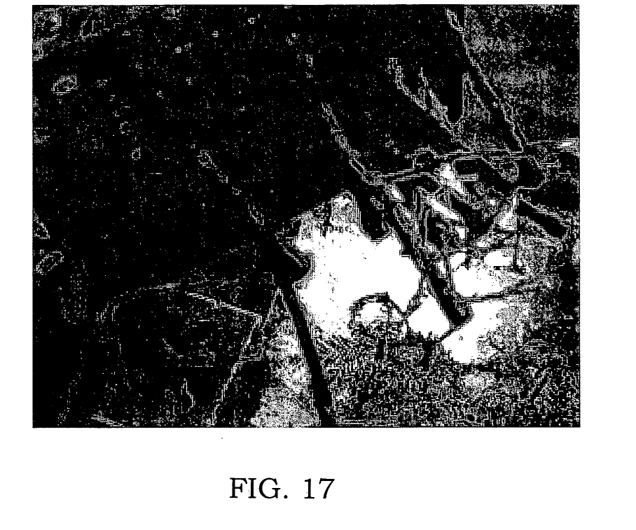

### BOTANICAL DESCRIPTION OF THE PLANT

The main botanical characteristics of ‘×Taxodiomera peizhongii’ follow:
Plant:
   Latin name.—×Taxodiomera peizhongii.
   Generic name.—Taxodium.
   Specific name.—×Taxodiomeria peizhongii.
   Plant height.—18 meters/30 year old tree.
   Canopy.—columnar coniform.
   Florescence.—Month of April.
   Optimal temperature.—15° C.-30° C.
   Seed and fruit productivity.—about 40-year-old trees have not been found to fruit.
   Characteristics.—tall indeciduous or semi-indeciduous arbor species.
   Habits: ×Taxodiomera peizhongii likes light, warm and humid climate, and wet soil; endures half-shade, moisture, salt alkali and impoverishment and resists wind well.
Stem or trunk and branch:
   Base.—round and regular.
   Diameter breast height.—55-62.5 cm for 40-year-old tree.
   Tapering grade.—small.
   Ramification characteristic.—there are crotches on the trunk.
   Bark color.—taupe.
   Bark crack.—be strip and broadways cracks into flakiness.
   Lateral branches.—stretch horizontally or upwards aslant.
   Persistent twigs.—grayish or henna and slippery, and with 1-1.5 mm diameter.
   Fallen twigs.—green.
   Distance among branches.—sparse.
   Twigs on big tree.—slender.
Foliage:
   Shape.—strip filate.
   Length.—3-10 mm.
   Width.—0.8-1 mm.
   Arrangement.—foliages on pleurogenous twigs array in two parallel rows, and the persistent twigs array parallel.
   Color.—dark greenish-black color.
   Stalk and basal.—short stalk and decurrent basal.
   Angle with the stem.—30 degree with pinnate foliage and 10-15 degree with twisted foliage.
   Venation.—midrib is greenish black.
   Thickness.—0.2-0.3 mm.
Fruit:
   Insertion manner of staminate flower.—the 2.5-3 mm round staminate flowers persist on the top of twigs.
   Female cone.—no female cone has been found.

This is a brand-new plant variety with adaptability characteristics that can grow well in all kinds of ecological conditions.

The plant claimed is ×Taxodiomera peizhongii.

The main botanical characteristics of the female parent of ‘×Taxodiomera peizhongii’ follow:
Plant:
   Latin name.—Taxodium mucronatum Ten.
   Generic name.—Taxodium.
   Specific name.—Taxodium mucronutun.
   Plant height.—50 meters in its native habitat.
   Canopy.—wide coniform.
   Florescence.—Month of April.
   Optimal temperature.—15° C.-30° C.
   Seed and fruit productivity.—This new variety can bear fruit in its native habitat, and cannot fruit without having completed its blooming period.
   Characteristics.—tall indeciduous or semi-indeciduous arbor species.
   Habits.—It likes light, warm and humid climate and wet soil. It has good endurance to moisture and resists wind.
Stem or trunk and branch:
   Base.—there are buttress roots on its base.
   Diameter breast height.—four meters in its native habitat.
   Tapering grade.—great.
   Ramification characteristic.—there are always crotches on its trunk.
   Bark color.—ficelle.
   Bark crack.—flaky strip.
   Lateral branches.—stretch upward aslant or horizontally.
   Persistent twigs.—dark taupe and slippery, and with 1.5-2 mm diameter.
   Fallen twigs.—green.
   Distance among branches.—moderate.
   Twigs on big tree.—slender.
   Shape.—strip filate.
   Length.—3-15 mm.
   Width.—1 mm.
Foliage:
   Arrangement.—foliages on pleurogenous twigs array in two parallel rows, and persistent twigs array parallel.
   Color.—bottle-green.
   Stalk and basal.—short stalk and decurrent basal.
   Angle with the stem.—30 degree for the pinnate foliages and 10-15 degree for the helical foliages.
   Venation.—midrib is greenish black.
   Thickness.—0.2-0.3 mm.
Fruit:
   Insertion manner of staminate flower.—2.5 mm-3 mm round staminate flowers persist on the top of twigs.
   Female cone.—solitary female cones persist on the top of twigs.
   Fruit stalk.—there is almost no stalk.
   Shape.—it is obovate round, and there are seminiferous scale enations on it.
   Color.—green to chocolate brown.
   Size.—15 mm-25 mm diameter.
   Maturation stage.—Months of October to November.
   Seminiferous scale.—ligneous and scutellate, two seeds on its ventral side.
Seed:
   Shape.—irregular triangle, and has obvious ridges.
   Color.—dark fulvous.
   Length.—4 mm-5 mm.

The main botanical characteristics of the male parent of ‘×Taxodiomera peizhongii’ follow:
Plant:
   Generic name.—Cryptomeria D. Don.
   Specific name.—Crytomeria fortunei.
   Plant height.—about 40 meters in its native habitat.
   Canopy.—columnar coniform.
   Florescence.—Month of April.
   Optimal temperature.—14° C.-25° C.
   Seed and fruit productivity.—it can fruit.
   Characteristics.—tall indeciduous arbor species.
   Habits.—it likes light, humid cool climate and loose soil with good drainability; and it endures half-shade and impoverishment.
Stem or trunk and branch:
   Base.—round and regular.
   Diameter breast height.—about 3 to 4 meters in its native habitat.
   Tapering grade.—small.
   Ramification characteristic.—erect trunk.
   Bark color.—taupe.
   Bark crack.—long strip flakiness.
   Lateral branches.—stretch horizontally or bend downwards.
   Persistent twigs.—reddish brown, have grooves under foliages and the diameter is about 2 mm-4 mm.
   Distance among branches.—moderate.
   Twigs on big tree.—thicker.
Foliage:
   Shape.—conical top end, and bend inwards.
   Length.—5 mm-15 mm.
   Width.—0.5 mm-1 mm.
   Arrangement.—foliages array in five rows helically.
   Color.—emerald.
   Stalk and basal.—short stalk and decurrent basal.
   Angle with the stem.—15 degree 30 degree.
   Venation.—midrib is not obvious, and there are pore lines around the midrib.
   Thickness.—0.8-1 mm.
Fruit:
   Insertion manner of staminate flower.—5-6 mm elliptical flowers persist on the top axillae of twigs.
   Female cone.—solitary female cone on top of twig or fasciculate cones.
   Fruit stalk.—there is almost no stalk.
   Shape.—it is near round with triangular enations on it.
   Color.—green to chocolate brown.
   Size.—diameter 12 mm-20 mm.
   Maturation stage.—Month of October.
Seed:
   Seminiferous scale.—ligneous and scutellate, and two seeds in it.
   Shape.—irregular triangle, and with narrow wing.
   Color.—puce.
   Length.—4 mm-5 mm.
   Width.—2 mm-3 mm.

1. A new and distinct variety of ×Taxodiomera peizhongii tree named ‘Dongfangshan’ as illustrated and described herein.
